SEQUENCE LISTING (22 sequences)

SEQ ID NO: 1 - DNA, 53 bp, Artificial Sequence
  misc_feature: Primer 1F
aattctaatacgactcactatagggagatgcttccaagattctggtgggaata

SEQ ID NO: 2 - DNA, 20 bp, Artificial Sequence
  misc_feature: Primer 1R
agtaagctaataatgatgat

SEQ ID NO: 3 - DNA, 35 bp, Artificial Sequence
  misc_feature: Primer 1S
aagcatacaatgtggagacaatgcataatcatcca

SEQ ID NO: 4 - DNA, 53 bp, Artificial Sequence
  misc_feature: Primer 2F
aattctaatacgactcactatagggagagcttttgattttaacgacttttaac

SEQ ID NO: 5 - DNA, 20 bp, Artificial Sequence
  misc_feature: Primer 2R
tgtagtgttgactggagcag

SEQ ID NO: 6 - DNA, 35 bp, Artificial Sequence
  misc_feature: Primer 2S
aaagcttgtcaattggaaccagtcgcaattatgaa

SEQ ID NO: 7 - DNA, 53 bp, Artificial Sequence
  misc_feature: Primer 3F
aattctaatacgactcactatagggagagaagctgtcatcggttactcagatt

SEQ ID NO: 8 - DNA, 20 bp, Artificial Sequence
  misc_feature: Primer 3R
cctcttctcgagagataccc

SEQ ID NO: 9 - DNA, 35 bp, Artificial Sequence
  misc_feature: Primer 3S
gcttcagccggaatttgtgccgtttcatcttctgt

SEQ ID NO: 10 - DNA, 53 bp, Artificial Sequence
  misc_feature: Primer 4F
aattctaatacgactcactatagggagattccggaagagccccctcagcaatg

SEQ ID NO: 11 - DNA, 21 bp, Artificial Sequence
  misc_feature: Primer 4R
ggactctctgggaatactggc

SEQ ID NO: 12 - DNA, 35 bp, Artificial Sequence
  misc_feature: Primer 4S
ccctccgggactgctaactggcaggagaacttctg

SEQ ID NO: 13 - DNA, 53 bp, Artificial Sequence
  misc_feature: Primer 5F
aattctaatacgactcactatagggagagagggagacagctctttctacatag

SEQ ID NO: 14 - DNA, 20 bp, Artificial Sequence
  misc_feature: Primer 5R
ggggtttctggccacggcag

SEQ ID NO: 15 - DNA, 35 bp, Artificial Sequence; identical to SEQ ID NO: 12
  misc_feature: Primer 5S

SEQ ID NO: 16 - DNA, 20 bp, Artificial Sequence
  misc_feature: Oligonucleotide Probe YO-3
cttctttagcagcaatgctg

SEQ ID NO: 17 - DNA, 20 bp, Artificial Sequence
  misc_feature: Oligonucleotide Probe AYO-3
cagcattgctgctaaagaag

SEQ ID NO: 18 - DNA, 180 bp, Artificial Sequence
  Human IL-6R-IL-6 Fusion Protein
tggatgattatgcattgtctccacattgtatgcttccaagattctggtgggaatactgctgatagcctaacgttcatgatcaaaatttaactgttctaacccctacttgacatcaatatataaacagaaggaagctgccctgtcttaaacctttttttttatcatcattattagcttact

SEQ ID NO: 19 - DNA, 180 bp, Artificial Sequence
  Human IL-6R-IL-6 Fusion Protein
ttcataattgcgactggttccaattgacaggcttttgattttaacgacttttaacgacaacttgagaagatcaaaaaacaactaattattcgaaggatccaaacgatgagatttccttcaatttttactgcagttttattcgcagcatcctccgcattagctgctccagtcaacactaca

SEQ ID NO: 20 - DNA, 180 bp, Artificial Sequence
  Human IL-6R-IL-6 Fusion Protein
acagaagatgaaacggcacaaattccggatgaagctgtcatcggttactcagatttagaaggggatttcgatgttgctgttttgccattttccaacagcacaaataacgggttattgtttataaatactactattgccagcattgatgataaagaagaaggggtatctctcgagaagagg

SEQ ID NO: 21 - DNA, 180 bp, Artificial Sequence
  Human IL-6R-IL-6 Fusion Protein
gttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcc

SEQ ID NO: 22 - DNA, 180 bp, Artificial Sequence
  Human IL-6R-IL-6 Fusion Protein
cagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccc

(19) United States
(12) Patent Application Publication
(10) Pub. No.: US 2003/0027142 A1
(43) Pub. Date: Feb. 6, 2003

(54) Novel genome analyzing method

(75) Inventors: Takahiko Ishiguro, Kanagawa (JP); Kiyoshi Yasukawa, Kanagawa (JP)

Correspondence Address: SUGHRUE MION ZINN MACPEAK & SEAS, PLLC, 2100 Pennsylvania Avenue, NW, Washington, DC, 20037-3213, US

(73) Assignee: TOSOH CORPORATION

(21) Appl. No.: 09/904,557

(22) Filed: Jul. 16, 2001

(30) Foreign Application Priority Data
Jul. 14, 2000 (JP) P. 2000-218737
Aug. 28, 2000 (JP) P. 2000-263248
Oct. 30, 2000 (JP) P. 2000-334935

(51) Int. Cl. C12Q 1/68; C12P 19/34

(52) U.S. Cl. 435/6; 435/91.2

(57) ABSTRACT

A novel transcriptome analyzing method and to provide a gene found by this method and a protein encoded by the gene. A method for determining whether or not a continued arbitrary DNA sequence existing in the genome of an arbitrary biological species, in which the nucleotide sequence is already known but its possibility of being a gene expression region is unclear (specific region), is the specific region, which comprises detecting whether or not a nucleotide sequence that corresponds to the nucleotide sequence of the region is present in the RNA of the biological species, and a method for determining the gene expression region in an arbitrary region on a genome or the entire genome, which comprises repeatedly carrying out the above method.

### FIELD OF THE INVENTION

This invention relates to a method for determining a gene expression region for a DNA sequence in which the nucleotide sequence is already known but its possibility of being a gene expression region is unclear (specific region) and a method for determining a gene expression region in an arbitrary region on a genome or the entire genome by repeatedly carrying out the above method. The invention also relates to a genomic gene which was determined to be a gene expression region by these methods and a protein encoded by the gene.

While nucleotide sequences of the genome in various biological species including the human genome composed of three billion bases are being revealed, development of so-called post-genome is in progress now. The target of post-genome is to understand kinds and activities of all proteins which are produced by a living thing during its entire life. Also, the main target for human post-genome is development of novel medicaments based on gene function analysis (creation of genomic drugs) and establishment of the basis of tailor-made medical treatments (cf., DeRisi et al., Science, vol. 278, p. 680, 1997).

In the post-genome, particularly the expression mode of RNA is called transcriptome. In transcriptome analysis, identification of all genes on the genome is an important subject. Even if a genomic DNA sequence is revealed, each gene is not identified.

The number of genes on the human genome is estimated to be one hundred thousand, but only six thousand have so far been revealed. Even if some of the remaining genes have important roles, it is difficult to identify them.

(1) For example, in a two-dimensional electrophoresis, rare proteins are easily lost among house-keeping proteins existing in large amounts, so that their discrimination is practically impossible. Also, analysis of cDNA libraries has the same problem; namely, a probability of selecting rare cDNA and subjecting it to nucleotide sequence determination is extremely small. What is more, when identification of all genes is the target, the degree of accomplishment at present cannot be known by these methods.

(2) For example, micro-alley is a technique to identify several thousand kinds of cDNA using tips to which they are linked, but since the linked cDNA molecules are already identified ones, conventionally unknown new genes are not identified.

(3) Also for example, an attempt to newly identify genes using a computer has been reported (cf., Bork et al., Nature Genet., vol. 18, p. 313, 1998), and programs such as GRAIL, HEXON and GENSCAN are provided for carrying out this method. However, it goes without saying that identification of genes based on not assumptions but experimental data is strongly expected in the transcriptome analysis.

Thus, the object of the invention is to provide a novel transcriptome analysis method and also to provide a gene found by such a method and a protein encoded by the gene.

### SUMMARY OF THE INVENTION

A first embodiment of the invention made for achieving the above object is a method for determining whether or not a continued arbitrary DNA sequence existing in the genome of an arbitrary biological species, in which the nucleotide sequence is already known but its possibility of being a gene expression region is unclear (specific region), is the specific region, which comprises detecting whether or not a nucleotide sequence that corresponds to the nucleotide sequence of the region is present in the RNA of the biological species.

A second embodiment of the invention relates to the first invention, wherein the specific region is a DNA region of from 100 to 200 bases.

A third embodiment of the invention relates to the first or second invention, wherein the detection is comprised of detecting whether or not DNA or RNA is amplified by the amplification of DNA or RNA based on the RNA of the biological species, using an oligonucleotide homologous to a sequence which is comprised of at least 10 or more continued bases and positioned in the 5′-end of the specific region and another oligonucleotide complementary to a sequence which is comprised of at least 10 or more continued bases and positioned in the 3′-end of the specific region.

A fourth embodiment of the invention relates to the third invention, wherein the amplification is an RNA amplification in which, using the oligonucleotides, either one of them having an RNA-transcriptable promoter sequence in its 5′-end, (1) a DNA fragment complementary to a part of RNA of the biological species is synthesized by RNA-dependent DNA polymerase from the either one of the oligonucleotides using the biological species-derived RNA as the template, thereby effecting formation of an RNA-DNA hybrid, (2) a single-stranded DNA fragment is formed by hydrolyzing the biological species-derived RNA of the RNA-DNA hybrid with ribonuclease H, (3) a DNA fragment complementary to the single-stranded DNA fragment is synthesized by DNA-dependent DNA polymerase from the other oligonucleotide using the single-stranded DNA fragment as the template, thereby effecting formation of a double-stranded DNA fragment having a promoter sequence capable of performing transcription of RNA as a part of the RNA of the biological species or RNA complementary to a part thereof, (4) an RNA transcription product is formed from the double-stranded DNA using RNA polymerase and then (5) the steps of from (1) to (4) are repeated using the RNA transcription product as the template.

A fifth embodiment of the invention relates to the third or fourth invention, wherein the detection of whether or not DNA or RNA is amplified is carried out by a method in which the amplification is carried out in the presence of an oligonucleotide probe which can specifically bind to the DNA or RNA formed by the amplification and is labeled with an intercalating fluorescence dye (provided that the oligonucleotide is a sequence which does not form complementary bonding with any one of the aforementioned oligonucleotides), and changes in a fluorescence characteristic of the reaction solution is measured.

A sixth embodiment of the invention relates to the fifth invention, wherein the probe can perform complementary bonding with at least a part of the sequence of the DNA transcription product or RNA transcription product formed by the amplification, and the fluorescence characteristic changes when compared with the case in which the complex is not formed.

A seventh embodiment of the invention is a method for determining the gene expression region in an arbitrary region on a genome or the entire genome, which comprises repeatedly carrying out the method of the first to sixth inventions.

An eighth embodiment of the invention is a genomic gene which was determined to be a gene expression region by the method of the first to seventh inventions. A ninth embodiment of the invention is a protein encoded by the gene of the eighth invention.

### BRIEF DESCRIPTION OF THE INVENTION

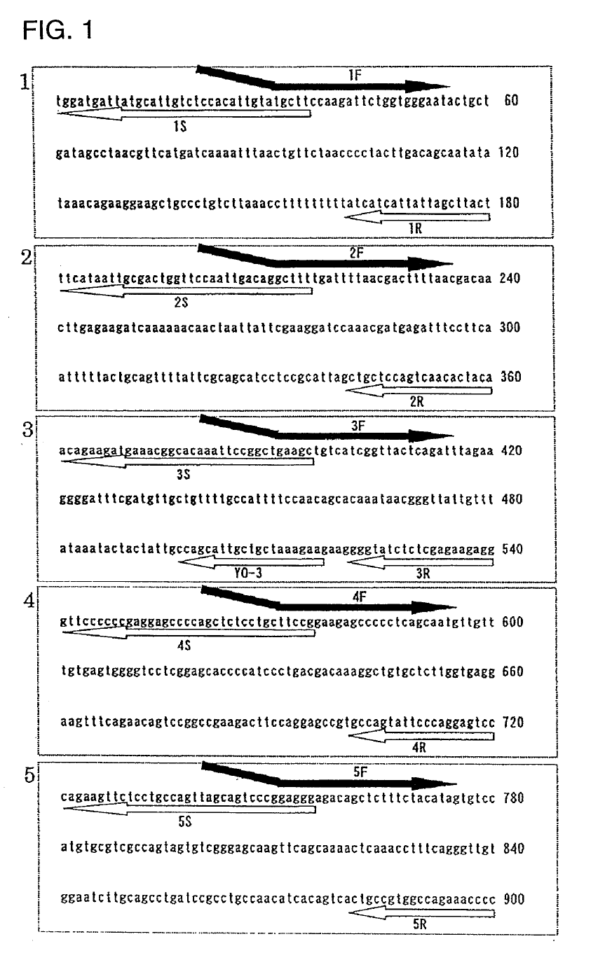

FIG. 1 shows a relationship between the nucleotide sequence of each of the specific regions 1 to 5 and the complementary bonding position of each of the primers 1F, 1R, 1S, 2F, 2R, 2S, 3F, 3R, 3S, 4F, 4R, 4S, 5F, 5R and 5S.

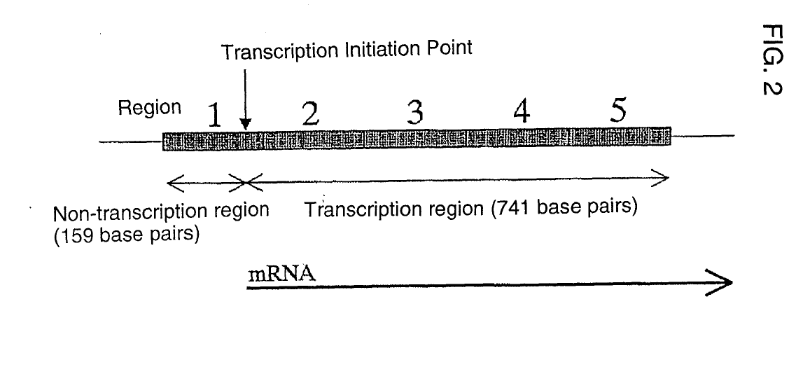

FIG. 2 shows the non-transcription region and transcription region of the specific regions 1 to 5.

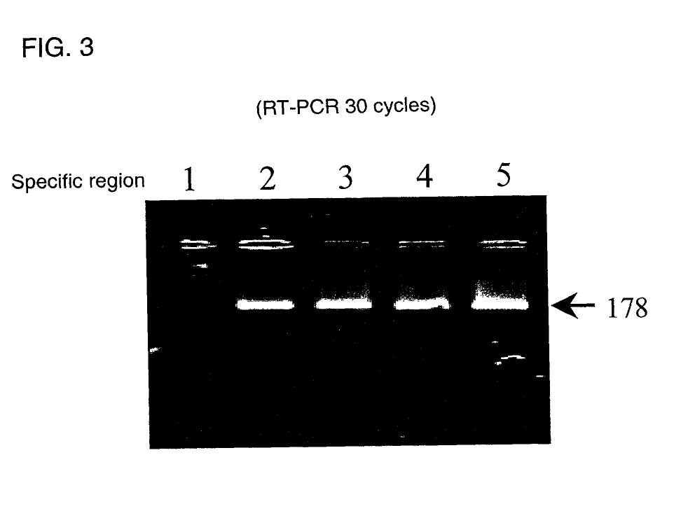

FIG. 3 shows an electrophoresis pattern when 30 cycles of RT-PCR was carried out for 200 ng of mRNA by the method shown in Example 3 using primers for the specific regions 1 to 5.

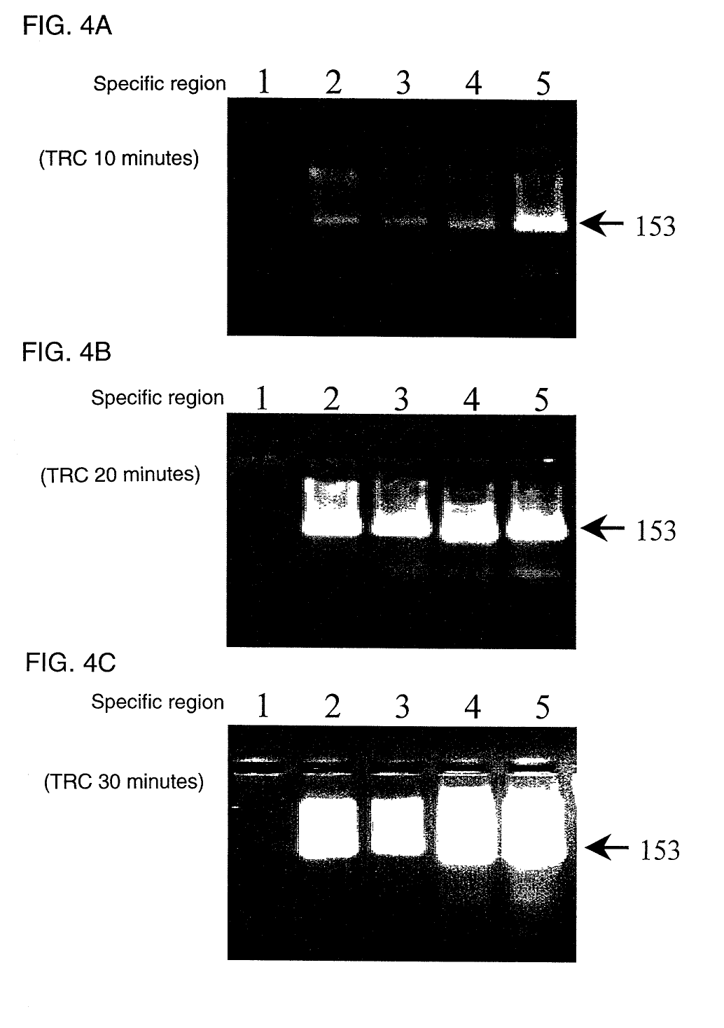

FIGS. 4A, 4B, 4C show respective electrophoresis patterns when 10, 20 and 30 minutes of TRC was carried out for 200 ng of mRNA by the method shown in Example 4 using primers for the specific regions 1 to 5.

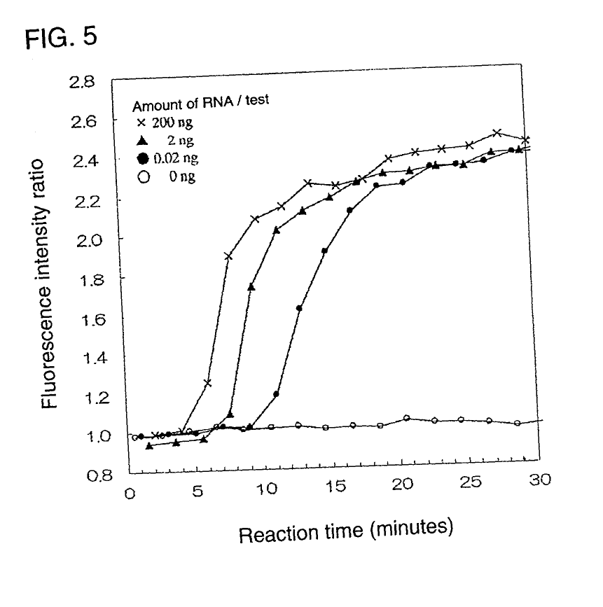

FIG. 5 is a graph showing a relationship between the reaction time and the fluorescence intensity ratio which increases with the formation of RNA, when TRC was carried out for 200 ng of mRNA by the method shown in Example 5 using primers for the specific region 3.

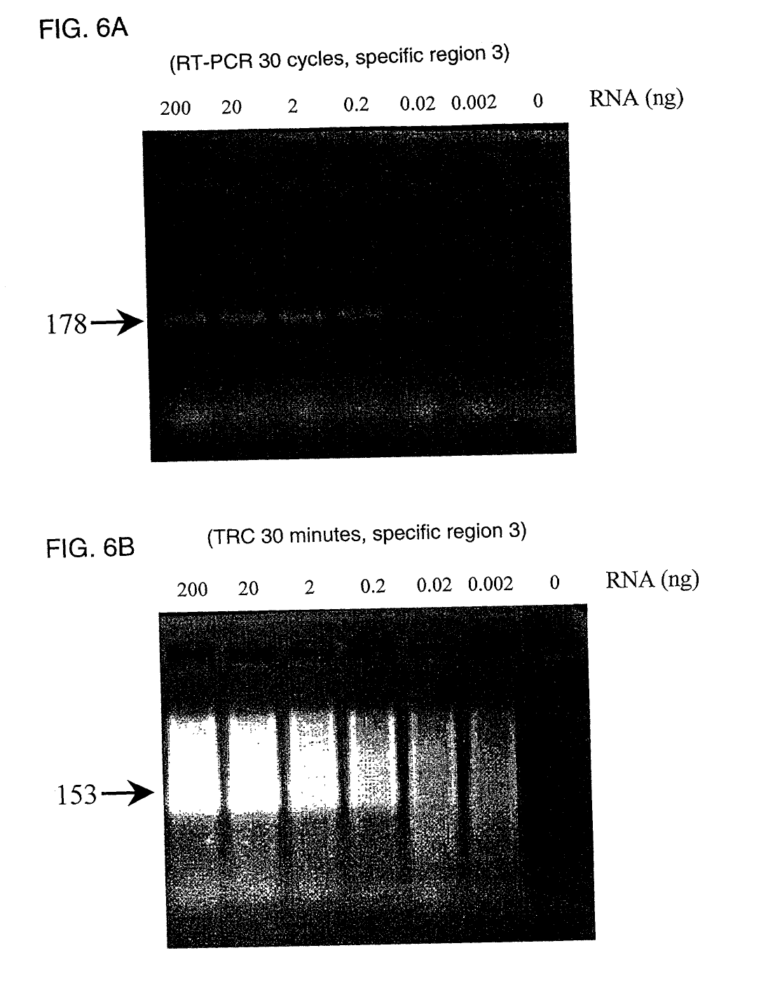

FIGS. 6A and 6B show respective electrophoresis patterns when 30 cycles of RT-PCR or 30 minutes of TRC was carried out for 200 ng of mRNA by the method shown in Example 6 using primers for the specific region 3.

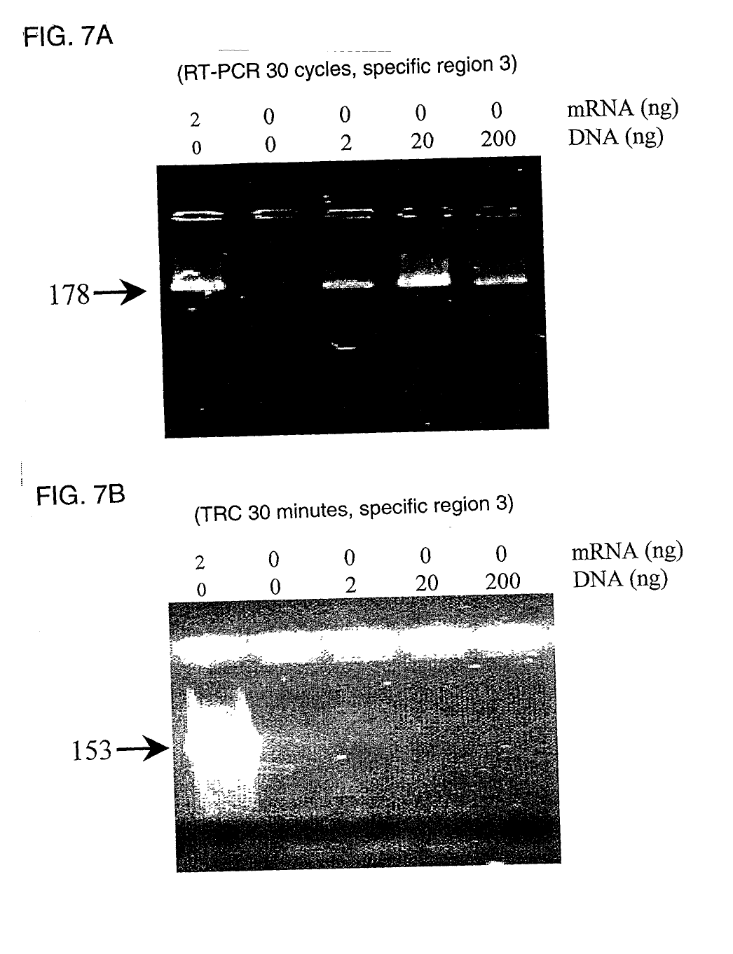

FIGS. 7A and 7B show respective electrophoresis patterns when 30 cycles of RT-PCR or 30 minutes of TRC was carried out for 0 or 2 ng of mRNA and 0 to 200 ng of genomic DNA, by the method shown in Example 7 using primers for the specific region 3.

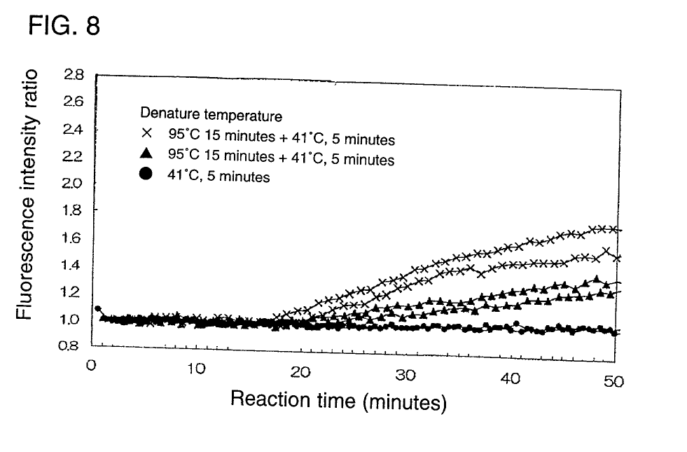

FIG. 8 is a graph showing a relationship between the reaction time and the fluorescence intensity ratio which increases with the formation of RNA, when TRC was carried out for 200 ng of genomic DNA by the method shown in Example 7 using primers for a region composed of the specific regions 1, 2 and 3.

### DETAILED DESCRIPTION OF THE INVENTION

The following describes the invention in detail.

The method of the invention is applied to a continued arbitrary DNA sequence existing in the genome of an arbitrary biological species, in which the nucleotide sequence is already known but its possibility of being a gene expression region is unclear (specific region). Such a specific region can be set by selecting from published genomic DNA sequences.

The length of the specific region is not particularly limited but is 200 bases or less, preferably within the range of from 100 to 200 bases. According to the method of the invention, a possibility that the specific region is a gene expression region can be determined only in a case in which the entire portion of an arbitrarily set specific region is included in one exon. Though the number of exons in a gene and the length of each exon greatly vary depending on the kind of gene, one exon containing a termination codon and a poly(A) connecting signal is present in every gene, which is longer than other exons and has more than 400 base pairs. Thus, when an arbitrary genomic region is fragmented into a specific region of 200 base pairs or less, at least one of the fragments is included in the exon and therefore is not overlooked.

According to the present invention, the following detection is carried out by using a continued arbitrary DNA sequence existing in the genome as the specific region, and the gene expression region can be determined on an arbitrary region in the genome or the entire genome by making the arbitrary region or the entire genome into fragments and repeating the detection using each fragment as the specific region.

The invention determines whether or not the specific region is a gene expression region by detecting the presence or absence of a nucleotide sequence which corresponds to the nucleotide sequence of the specific region, in RNA of the same biological species. The RNA to be used in the invention is mRNA which is prepared from the same biological species containing the genome to be determined for the gene expression region. Particularly, when the genome is a genome of a higher organism, it is desirable to use various types of mRNA, preferably prepared from all tissues. In that case, the mRNA may be used separately for each tissue or mixed. When the presence of a gene expression region is found in the latter case, subsequent separate use of mRNA of each tissue renders possible finding of a tissue which is expressing the gene in the genome determined to be the gene expression region. The reason that mRNA species can be mixed is as follows. Assuming that average molecular weight of mRNA is 300,000, 1 ng of mRNA will contain 2×10⁹ mRNA molecules. Accordingly, even in the case of a gene which is expressed in only one of 1,000 tissues and its expressing quantity is in a ratio of 1/100,000 of mRNA in the tissue, 2×10⁴ copies are present in 1 μg of the same amount mixture of mRNA respectively obtained from 1,000 tissues including this tissue. As will be described later in Examples, this copy number is sufficiently detectable.

Various method can be applied to the above detection. For example, application of a hybridization method and a nucleic acid amplification method can be exemplified. When an amplification method is used, at least two oligonucleotides (primers) designed based on the specific region are used in both DNA amplification and RNA amplification, and one of them is an oligonucleotide homologous to a sequence which is comprised of at least 10 or more continued bases and positioned in the 5′-end of the specific region and the other is an oligonucleotide complementary to a sequence which is comprised of at least 10 or more continued bases and positioned in the 3′-end of the specific region. Oligonucleotides of at least 10 or more bases are used for keeping a specificity regarding binding of the oligonucleotides to the specific region.

Examples of the nucleic acid amplification method include a DNA amplification method typified by RT-PCR in which cDNA is synthesized from the mRNA using primers and a reverse transcriptase and then DNA (DNA comprised of the specific region) is amplified by a primer elongation reaction using the primers and a DNA polymerase and the DNA as the template, and an RNA amplification method in which cDNA complementary to the RNA is synthesized using primers and a reverse transcriptase and the mRNA as the template, an elongation reaction of DNA is carried out by binding it to a promoter primer having a moiety complementary to the DNA and then RNA (RNA comprised of the specific region) is synthesized in a large amount by allowing an RNA polymerase to react with the thus synthesized double-stranded DNA. The former case is an already broadly and generally known method, and examples of the latter case include NASBA (nucleic acid sequence based amplification) method, 3SR method and the method which will be described later in Examples.

In describing outlines of the NASBA method and the method described in Examples, they are RNA amplification in which, using the oligonucleotides, either one of them having an RNA-transcriptable promoter sequence in its 5′-end, (1) a DNA fragment complementary to a part of RNA of the biological species is synthesized by RNA-dependent DNA polymerase from the either one of the oligonucleotides using the biological species-derived RNA as the template, thereby effecting formation of an RNA-DNA hybrid, (2) a single-stranded DNA fragment is formed by hydrolyzing the biological species-derived RNA of the RNA-DNA hybrid with ribonuclease H, (3) a DNA fragment complementary to the single-stranded DNA fragment is synthesized by DNA-dependent DNA polymerase from the other oligonucleotide using the single-stranded DNA fragment as the template, thereby effecting formation of a double-stranded DNA fragment having a promoter sequence capable of performing transcription of RNA as a part of the RNA of the biological species or RNA complementary to a part thereof, (4) an RNA transcription product is formed from the double-stranded DNA using RNA polymerase and then (5) the steps of from (1) to (4) are repeated using the RNA transcription product as the template.

The method described in Examples can be exemplified as particularly desirable detection method from the viewpoints that the determination of the invention can be effected within a short period of time because the amplification is completed within a markedly short time of 10 minutes, that it has a high sensitivity which enables amplification of even several pg of RNA containing the specific region and that the influence of DNA having a possibility of contaminating RNA can be excluded.

The DNA and RNA formed by the above amplification can be detected by an already known detection method such as an electrophoresis, but particularly preferred is a method in which the amplification is carried out in the presence of an oligonucleotide probe which can specifically bind to the DNA or RNA formed by the amplification and is labeled with an intercalating fluorescence dye, and changes in a fluorescence characteristic of the reaction solution is measured. As a matter of course, this probe is a sequence which does not form complementary bonding with the oligonucleotides used in the amplification. Examples of this oligonucleotide probe include those in which an intercalating fluorescence dye is linked to the phosphorus of an oligonucleotide via a linker. In the case of such a suitable probe, when the formed DNA or RNA forms double-strand by complementary bonding to a specific region (or a sequence complementary to the specific region), the intercalating fluorescence dye intercalates into the double-stranded moiety and changes its fluorescence characteristic, so that it is not necessary to separate the probe which did not form complementary bonding (Ishiguro, T. et al., (1996), Nucleic Acids Res., 24 (24), 4992-4997).

Nucleotide sequence of the oligonucleotide probe is not particularly limited with the proviso that it has a sequence that can perform complementary bonding with the formed DNA or RNA, but in order to keep a specificity regarding its bonding to the formed DNA or RNA, it is desirable that it has about 10 bases which are complementary to at least 10 continued bases existing in the DNA or RNA. In this connection, when the amplification is carried out in the presence of the oligonucleotide probe, it is desirable to modify the hydroxyl group of the 3′-end of the probe chemically (e.g., addition of glycolic acid) in order to suppress elongation reaction in which the probe is used as a primer.

When the amplification is carried out in the presence of the oligonucleotide probe as described above, the detection process of the invention can be carried out in one reaction container at a constant temperature and by one step, so that its application to automatic operation can be made easily.

Details of the genome analysis method of the invention which is carried out by repeating the gene expression region determination method are as follows, and the method can be applied to any biological species if the genomic sequence is determined. The genome of said biological species is divided, for example, into specific regions each having 200 base pairs. When a nucleic acid amplification is used as the detection method, a primer set containing two oligonucleotides necessary for amplifying each specific region is prepared. In this connection, the number of necessary primers and their sequences vary depending on the nucleic acid amplification method to be employed. Also, it is effective for improving working efficiency to exclude a specific region which is present in a region already known as a gene expression region by previous studies and a specific region which is present in a region that is obviously not a gene expression region based on its DNA sequence, from the objects. Next, the RNA is detected using a primer set for each specific region.

When a genome is analyzed by the method of the invention, all genes of the biological species can be identified. In addition, it becomes possible to determine a protein encoded by a gene of interest, by isolating the gene determined to be a gene expression region and to produce the protein making use of the isolated DNA. For example, a nucleotide sequence can be determined by isolating complete length cDNA in the usual way using a nucleic acid amplified by the method of the invention as a probe. By doing this, the genomic structure including the relationship between intron and exon in the gene expression region is revealed. Also, a protein encoded by the gene can be known by isolating cDNA through the screening of a cDNA library in the usual way using the amplified nucleic acid as a probe. In addition, when this protein is expressed, it can be expressed by preparing a recombinant using the cDNA and using a microbial or animal cell as the host in the usual way.

Examples of the invention are given below by way of illustration and not by way of limitation.

### EXAMPLE 1

Establishment of Regions

In order to show realization possibility of the gene expression region determination method provided by the invention, the following model test was carried out.

As the genomic region, a region composed of 900 base pairs prepared from a GI strain, a genetically engineered transformed methanol assimilating yeast strain which has been established by the method described by the present inventors in Japanese Patent Application No. 11-188650, was selected. When induced by methanol, the GI strain expresses a human IL-6R-IL-6 fusion protein composed of one polypeptide chain of 397 amino acid residues (cf., Japanese Patent Application No. 11-188650).

As shown in FIG. 1, the region composed of 900 base pairs was divided into five specific regions each having 180 base pairs. Also, mRNA expressing mode of the region already known from Japanese Patent Application No. 11-188650 is shown in FIG. 2. As is evident from FIGS. 1 and 2, the specific region 1 (base numbers 1 to 180) contains 159 base pairs of a non-transcription region and 21 base pairs of a transcription region. Each of the specific region 2 (base numbers 181 to 360), specific region 3 (base numbers 361 to 540), specific region 4 (base numbers 541 to 720) and specific region 5 (base numbers 721 to 900) contains only a transcription region.

Oligonucleotide (primer) sets (forward primer; F, reverse primer; R, scissor probe; S) shown in FIG. 1 and SEQ ID NOs;1 to 15 were synthesized for each of the above five specific regions. When DNA amplification (RT-PCR) was carried out, the forward primer and reverse primer among them were used. When RNA amplification (TRC; transcription reverse transcription concerting amplification) was carried out, the forward primer, reverse primer and scissor probe were used. In the TRC, a specific region cannot be amplified when it is not located at the 5′-terminal of mRNA. The scissor probe is an oligonucleotide (DNA) to be used in that case for locating the specific region to the 5′-side of mRNA by complementarily binding it to the 5′-side of the specific region and cutting the complementarily bonded region by the action of a ribonuclease.

### EXAMPLE 2

Preparation of mRNA

An mRNA sample of the strain G1 was prepared by the following method.

The strain was inoculated into 3 ml of BMGY (Bacto Yeast Extract 10 g/l, Bacto Peptone 20 g/l, Yeast Nitrogen Base without amino acids 1.34 g/l, 100 mM potassium phosphate buffer, pH 6.0, glycerol 10 g/l and biotin 0.4 mg/l) medium, and cultured at 28° C. for 24 hours on a shaker.

A 100 μl portion of the culture broth was inoculated into 3 ml of BMGY (Bacto Yeast Extract 15 g/l, Bacto Peptone 30 g/l and other components having the same composition of the above BMGY) medium, and cultured at 28° C. for 16 hours.

After confirmation of the depletion of methanol, 100 μl of methanol was added to the medium to induce expression of the human IL-6R-IL-6 fusion protein. Two hours after the addition of methanol, the cells were collected and 5×10⁷ of the cells were immediately frozen with liquid nitrogen.

They were subjected to cell wall lysis using a commercially available kit (Yeast cell lysis preparation kit, mfd. by BIO 01 Inc.) Next, mRNA was prepared using a commercially available kit (QuickPrep mRNA Purification Kit, mfd. by Amersham Pharmacia).

### EXAMPLE 3

Determination of Gene Expression Region by DNA Amplification

Using the mRNA obtained in Example 2, examination was carried out on whether or not the DNA amplification is specific for a primer derived from a region composed solely of a gene expression region.

A commercially available kit (RT-PCR beads, mfd. by Amersham Pharmacia) was used in the RT-PCR.

That is, cDNA was synthesized from 200 ng of mRNA by a 15 minutes of reaction at 42° C. using oligo(dT) as a primer. Next, PCR reaction was carried out using the forward primer and reverse primer. Using a thermal cycler, a cycle composed of 95° C. for 1 minute, 55° C. for 1 minute and 72° C. for 2 minutes was repeated 30 cycles spending about 3 hours. Immediately after the reaction, an electrophoresis was carried out using 4% agarose which was then stained with SYBR Green.

As is evident from FIG. 3, amplification was not found by the primer originated from the specific region 1 but was found by the primers originated from the specific regions 2 to 5.

These results show that the DNA amplification is specific for a primer derived from a region composed solely of a gene expression region, that is, whether or not a continued arbitrary DNA sequence existing in the genome of an arbitrary biological species, in which the nucleotide sequence is already known but its possibility of being a gene expression region is unclear (specific region), is a gene expression region can be determined by detecting the presence or absence of a nucleotide sequence which corresponds to the nucleotide sequence of the region in the RNA of the biological species, by a DNA amplification typified by RT-PCR.

### EXAMPLE 4

Determination of Gene Expression Region by RNA Amplification

Using the mRNA obtained in Example 2, examination was carried out on whether or not the RNA amplification is specific for a primer derived from a region composed solely of a gene expression region. (1) Using an RNA dilution solution (10 mM Tris-HCl (pH 8.0) and 1 mM EDTA), the sample was diluted to 200 ng/5 μl.

(2) A 20.8 μl portion of a reaction solution of the following composition was dispensed into 0.5 ml capacity tubes and 5 μl of the above RNA sample was added thereto.

Reaction solution composition (each concentration is a concentration in 30 μl of the final reaction solution)

60 mM of Tris-HCl (pH 8.6),

13 MM of MgCl₂,

90 mM of KCl,

39 U of RNase inhibitor,

1 mM of DTT,

0.25 mM of each of DATP, dCTP, dGTP and gTTP,

3.6 mM of ITP,

3.0 mM of each of ATP, CTP, GTP and TTP,

0.16 μM of scissor probe,

1 μM of forward primer,

1 μM of reverse primer,

13% of DMSO, and

distilled water for volume adjustment.

(3) This reaction solution was incubated at 65° C. for 15 minutes and then at 41° C. for 5 minutes, and then 4.2 μl of an enzyme solution having the following composition was added thereto.

Enzyme solution composition (each concentration is a concentration in 30 μl of the final reaction solution)

1.7% of sorbitol,

3 μg of bovine serum albumin,

142 U of T7 RNA polymerase (mfd. by Gibco),

8 U of AMV reverse transcriptase (mfd. by Takara Shuzo),

distilled water for volume adjusting use.

(4) Subsequently, the tubes were kept at 41° C. for 10, 20 or 30 minutes. Immediately after the reaction, an electrophoresis was carried out using 4% agarose which was then stained with Cyber Green.

As is evident from FIGS. 4A, 4B and 4C, amplification was not found when the primer for the specific region 1 was used in each case of the 10 minute reaction (FIG. 4A), 20 minute reaction (FIG. 4B) and 30 minute reaction (FIG. 4C), but was found when the primers for the specific regions 2 to 5 were used.

These results show that the RNA amplification is specific for a primer derived from a region composed solely of a gene expression region, that is, whether or not a continued arbitrary DNA sequence existing in the genome of an arbitrary biological species, in which the nucleotide sequence is already known but its possibility of being a gene expression region is unclear (specific region), is a gene expression region can be determined by detecting the presence or absence of a nucleotide sequence which corresponds to the nucleotide sequence of the region in the RNA of the biological species, by an RNA amplification typified by TRC.

Also, while the RT-PCR amplification shown in Example 3 required 3 hours even by the use of a thermal cycler, 10 minutes were enough for the amplification by TRC.

### EXAMPLE 5

Measurement Using Oligonucleotide Probe Labeled with Intercalating Fluorescence Dye

Using the mRNA obtained in Example 2, measurement using an oligonucleotide probe labeled with an intercalating fluorescence dye was carried out.

(1) Using an RNA dilution solution (10 mM Tris-HCl (pH 8.0) and 1 mM EDTA), the sample was diluted to 200 ng/5 μl.

(2) A 20.8 μl portion of a reaction solution of the following composition was dispensed into 0.5 ml capacity tubes and 5 μl of the above RNA sample was added thereto.

Reaction solution composition (each concentration is a concentration in 30 μl of the final reaction solution)

60 mM of Tris-HCl (pH 8.6),

13 mM of MgCl₂,

90 mM of KCl,

39 U of RNase inhibitor,

1 mM of DTT,

0.25 mM of each of DATP, dCTP, dGTP and gTTP,

3.6 mM of ITP,

3.0 mM of each of ATP, CTP, GTP and TTP,

0.16 μM of scissor probe (3S, SEQ ID NO;9, the

3′-terminal hydroxyl group is aminated),

1 μM of forward primer (3F, SEQ ID NO;7),

1 μM of reverse primer (3R, SEQ ID NO;8),

25 nM of an oligonucleotide labeled with an intercalating fluorescence dye (YO-3, SEQ ID NO;16, the intercalating fluorescence dye is labeled on the phosphorus between 6th position “T” and 7th position “T” counting from the 5′-terminal, and the 3′-terminal hydroxyl group is modified with glycol group),

13% of DMSO, and

distilled water for volume adjustment.

(3) This reaction solution was incubated at 65° C. for 15 minutes and then at 41° C. for 5 minutes, and then 4.2 μl of an enzyme solution having the following composition was added thereto.

Enzyme solution composition (each concentration is a concentration in 30 μl of the final reaction solution)

1.7% of sorbitol,

3 μg of bovine serum albumin,

142 U of T7 RNA polymerase (mfd. by Gibco),

8 U of AMV reverse transcriptase (mfd. by Takara Shuzo)

distilled water for volume adjusting use.

(4) Subsequently, the tubes were kept at 41° C. and the reaction solution was periodically measured at an excitation wavelength of 470 nm and a fluorescence wavelength of 510 nm using a directly measurable fluorescence spectrophotometer equipped with a temperature controlling function.

Periodical changes in the fluorescence intensity ratio of the sample (fluorescence intensity value at a predetermined time/background fluorescence intensity value) calculated by defining the time of the enzyme addition as 0 minute are shown in FIG. 5.

As shown in FIG. 5, the target RNA contained in 200 ng of mRNA was detected within about 6 minutes. In addition, the target RNA was detected within about 11 minutes even when the amount of mRNA was reduced to 0.02 ng. Thus, it was shown that quick and high sensitivity measurement can be made by the use of an oligonucleotide probe labeled with an intercalating fluorescence dye.

### EXAMPLE 6

Sensitivity

Sensitivities of RT-PCR and TRC were compared.

Using from 0 to 200 ng of the mRNA obtained in Example 2, amplification of DNA or RNA was carried out by 30 cycles of RT-PCR by the method shown in Example 3 or by 30 minutes of TRC by the method shown in Example 4.

As is evident from FIGS. 6A and 6B, amplification of 0.002 ng of the mRNA was not detected by RT-PCR but was detected by TRC. Thus, it was shown that TRC can achieve 10 times higher sensitivity than RT-PCR.

### EXAMPLE 7

Influence of DNA Contamination

Influences of the contamination of mRNA with DNA in RT-PCR and TRC were examined.

Firstly, using a commercially available kit (G Nome, mfd. by BIO 101 Inc.), genomic DNA was prepared from the cell wall-lysed G1 cell strain obtained by the method described in Example 1. Using from 0 to 200 ng of the DNA and 0 or 200 ng of the mRNA obtained in Example 2, 30 cycles of RT-PCR was carried out by the method shown in Example 3, and 30 minutes of TRC by the method shown in Example 4. As is evident from FIGS. 7A and 7B, amplification was observed by RT-PCR when from 2 to 200 ng of the genomic DNA was present even in the absence of the mRNA. On the other hand, the amplification did not occur by TRC in the absence of the mRNA even when from 2 to 200 ng of the genomic DNA was present.

Next, relationship between denaturing condition and amplification of genomic DNA in TRC was examined. Using 200 ng of the genomic DNA, measurement by an oligonucleotide probe (YO-3, SEQ ID NO;16) labeled with an intercalating fluorescence dye was carried out by the method shown in Example 5. In this case, IS (SEQ ID NO;3) was used instead of 3S (SEQ ID NO;3) as the scissor probe, and 1F (SEQ ID NO;1) was used instead of 3F (SEQ ID NO;7) as the forward primer. The reason for changing the scissor probe and forward primer is to prevent generation of amplification from RNA by changing the amplifying region to a region of 540 base pairs composed of the specific regions 1, 2 and 3 containing the 159 base pair non-transcription region. Also, the constant treating condition of the reaction solution before addition of the enzyme solution (incubation at 65° C. for 15 minutes and then at 41° C. for 5 minutes) was changed to the following three conditions.

(1) Incubation at 95° C. for 15 minutes and then at 41° C. for 5 minutes

(2) Incubation at 65° C. for 15 minutes and then at 41° C. for 5 minutes

(3) Incubation at 41° C. for 5 minutes

As is evident from FIG. 8, the time when the fluorescence intensity ratio exceeded 1.2 was about 28 minutes under the condition (1) and about 40 minutes under the condition (2), but its increase was not found under the condition (3).

This result shows that amplification can also occur from DNA by strengthening the denaturing condition. In this case, a change of the treating condition of the reaction solution before addition of the enzyme solution for the condition (3) is convenient in inhibiting amplification from DNA. However, it is expected that the amplification from RNA will be inhibited due to formation of the secondary structure of RNA. In addition, since periodical changes in the fluorescence intensity ratio are greatly different between the amplification from RNA and the amplification from DNA, it is markedly easy to make distinctions between both cases by comparing FIG. 5 with FIG. 8.

In summing up the above results, it is considered that a condition of incubating at 65° C. for 15 minutes and then at 41° C. for 5 minutes is appropriate as the treating condition of the reaction solution before addition of the enzyme solution in the mode for carrying out the invention.

Since the use of the method provided by the invention renders possible revelation of gene expression regions in the entire genome and also of the genomic structure including the intron-exon relation ship, sequences of all proteins capable of being expressed in an arbitrary biological species can be easily determined.

Consequently, according to the present invention, it is considered that understanding of all vital phenomena becomes possible by making rapid progress in the post-genome. Also, it is expected that the human post-genome will lead to the development of novel therapeutic and diagnostic drugs and also will greatly contribute to the progress of order-made medical treatments. It also will greatly contribute to the identification of industrially useful proteins from microorganisms living under extreme environmental conditions and to the application thereof.

While the invention has been described in detail and with reference to specific embodiments thereof, it will be apparent to one skilled in the art that various changes and modifications can be made therein without departing from the spirit and scope thereof.

This application is based on Japanese patent applications No. 2000-218737 filed on Jul. 14, 2000, No. 2000-263248 filed on Aug. 28, 2000 and No. 2000-334935 filed on Oct. 30, 2000, the entire contents of each of which are hereby incorporated by reference.

What is claimed is:

1. A method for determining whether or not a continued arbitrary DNA sequence existing in the genome of an arbitrary biological species is the specific region, wherein said nucleotide sequence is known but its possibility of being a gene expression region is unclear (specific region), which comprises:
detecting whether or not a nucleotide sequence that corresponds to the nucleotide sequence of said region is present in the RNA of said biological species.

2. The method according to claim 1, wherein said specific region is a DNA region of from 100 to 200 bases.

3. The method according to claim 1 or 2, wherein said detection comprises detecting whether or not DNA or RNA is amplified by carrying out amplification of DNA or RNA based on the RNA of said biological species, using an oligonucleotide homologous to a sequence which is comprised of at least 10 or more continued bases and positioned in the 5′-end of said specific region and another oligonucleotide complementary to a sequence which is comprised of at least 10 or more continued bases and positioned in the 3′-end of said specific region.

4. The method according to claim 3, wherein at least one of said oligonucleotides has an RNA-transcriptable promoter sequence in its 5′-end and said amplification is an RNA amplification comprising:
(1) synthesizing a DNA fragment complementary to a part of RNA of said biological species by RNA-dependent DNA polymerase from said either one of the oligonucleotides using said biological species-derived RNA as the template, thereby effecting formation of an RNA-DNA hybrid,
(2) forming a single-stranded DNA fragment by hydrolyzing the biological species-derived RNA of said RNA-DNA hybrid with ribonuclease H,
(3) synthesizing a DNA fragment complementary to said single-stranded DNA fragment by DNA-dependent DNA polymerase from the other oligonucleotide using the single-stranded DNA fragment as the template, thereby effecting formation of a double-stranded DNA fragment having a promoter sequence capable of performing transcription of RNA as a part of the RNA of said biological species or RNA complementary to a part thereof,
(4) forming an RNA transcription product from said double-stranded DNA using RNA polymerase, and
(5) the repeating the steps of from (1) to (4) using said RNA transcription product as the template.

5. The method according to claim 3 or 4, wherein said detection of whether or not DNA or RNA is amplified is carried out by a method comprising:
carrying out the amplification in the presence of an oligonucleotide probe which can specifically bind to the DNA or RNA formed by the amplification and is labeled with an intercalating fluorescence dye, provided that said oligonucleotide is a sequence which does not form complementary bonding with any one of the aforementioned oligonucleotides, and
measuring the change in a fluorescence characteristic of the reaction solution.

6. The detection method according to claim 5, wherein said probe is capable of performing complementary binding with at least a part of the sequence of the DNA transcription product or RNA transcription product formed by the amplification to change the fluorescence characteristic as compared with the case in which the complex is not formed.

7. A method for determining the gene expression region in an arbitrary region on a genome or the entire genome, which comprises repeatedly carrying out the method of any one of claims 1 to 6.

8. A genomic gene which was determined to be a gene expression region by the method of any one of claims 1 to 7.

9. A protein encoded by the gene of claim 8.
